SEQUENCE LISTING (6 sequences)

SEQ ID NO: 1 - DNA, 780 bp, Artificial
  upstream region of AFPalphaNeoTK plasmid
  misc_feature (758)..(780): n is A, T, C, or G.
aattcaggtggctcacagcatactaggatgtgagggcgcatgggaagcaccgtgaggaaacgatctaaagagcgttagacgttgtcagagagggacgttctccagtgagcacttgggtacacacacagacaattatatgatctgtaccttgaagagtagtggtaagatttgataagcattgatgggtaaggcactagaaatgtaagaactgtgctttcagggacacatatttgcacacacacacacacacacacacacacacaaaggatcctgaaagagtgaaagtcattttctgctaactcacatgtgcagtttaactgtacattttaaatataccagtcctaagaatggtgatatttactctcatctttttagaaatatttgtagccacccagcacttaggatacatcattccacctccacacgaatgtacacttgaggagaggtattgtttctgtgatacaaatgataaaatccagattcagtcatattacattatttgctgaatgtcgtatggcaagcaacgggcacaagcaagattagcatctttttgatggcagagttcagtttaccgggtcacattgtacctgggaagattcaaggatttatggaaaaagtcaacaacaggagtcagagcagcccggaaaagcatggactctgtacttaaggactgcgcttttgagcaatggcacagcaagctttaaccctgtttgcagtcagcacacaaactgtgggtcaaaagcttccactttatcttnnnnnnnnnnnnnnnnnnnnnnn

SEQ ID NO: 2 - DNA, 10460 bp, Artificial
  Downstream region of AFPalphaNeoTK plasmid
  misc_feature (1)..(45): n is A,T, C, or G.
nnnnnnnnnnnnnnnnnnnnnnnnnnnnnnnnnnnnnnnnnnnnnttatcaacactttattttgaggacagaaagaaagtgcaggtacagtttgaaaatatggtcatactggggtgtttgttttcccattttctattactaatatattatgggaatctttccattgcattaaatgttactctatgaactaggtttgatcgtagtactcccctccattttatgagtacactatattttctagtgtaagttatttgtttttataatcacatctttaacataaccgctcctaggctctgcgatggctcagtagttaagggcacctttctgctcttccagaggacctgggttcaattttcagtgcccacatggcagctcacaaccatctgtaactctagttccaggggatccaatatcctcttcagacctcttcaggaaacagctatgcacatagcacacaggcatatgttcaaccaaaacactgaaacacataaaagaaatgtttaaagaatgaatttaaaaaaataaaaaataaactcaactacatatgaagccttagcaaacatgtctggacctctagacacacagactctgacacgccaacgtctgagttctagtttcgatacgcactgggaagttttaaaagttttccatcaactctaatgtgtagagaaatggaaactatcatagactctacggcattgagggtgaaggtatgagtgaagcactcttagggtcagaagtatgtcagtgcccatttgttgctgttagcatcatcatcttagggcttgagaggatgttgcagctgacccatgcacctgtgacatacatatggaattattctttggcacataaaattagaatgggagctggctcatcaggttttgtgctgtaagttttctatgttaaaccagatgcgatacactaaataaaataaaatatacttgaccgatggttttgagcgaaataataactggataatcaagaaatatatccactaatgaatagcctgactactgaaacaatttgttcagtgcctagcatatggtgtgcattttattatttctttcaaaaagaatgtatttggagttacatagtaagtctgctaccttttctttatggctatatctatgtcttatgttgagatgaatgaattattcttcaggggaaataatctatttgaacagtttagatggtgaagaacatttgcagcatttgcaagatttttttccactctgaagtggtctttgtccttgaacataggatacaagtgacccctgctctgttaattattggcaaattgcctaacttcaacgtaaggaaatagagtcatatgtttgctcactgaaggttactagttaacaggcatcccttaaacaggatataaaaggacttcagcaggactgctcgattcgcgacccgaagctgcgcgggcgcgagccagttggggcactgggtgggcggcggcgacagcggcgccacgcgcaggctggaggccgccgaggctcgccatgccgggagaactctaactcccccatggagtcggccgacttctacgaggtggagccgcggcccccgatgagcagtcacctccagagccccccgcacgcgcccagcaacgcccgcctttggctttccccggggcgcgggccccgcgccgcccccagccccacctgccgccccggagccgctgggcggatctgcgagcacgagacgtctatagacatcagcgcctacatcgacccggccgccttcaacgacgagttcctggccgacctcttccagcacagccgacagcaggagaaggccaaggcggcggcgggccccgcgggtggcggcggtgactttgactacccgggagccccggcgggccccggcggcgcggtcatgtccgcgggggcgcacgggccccctcccggctacggctgtgcggcggccggctacctggacggcaggctggagcccctgtacgagcgcgtcggggcgcccgcgctacggccgctggtgatcaaacaagagccccgcgaggaggacgaggcgaagcagctggcgctggccggcctcttcccctaccagccaccgccgccaccgccaccgccgcacccgcacgcgtctcccgcgcacctggccgccccccacttgcagttccagatcgcgcactgcggccagaccaccatgcacctacagcctggccaccccacaccgccgcccacgcccgtgcccagcccgcacgctgcgcccgccttgggtgctgcgggcctgcctggccccgggagcgcgctcaagggcttggccggtgcgcaccccgacctccgcacgggaggcggcggcggtggcagcggtgccggtgcgggcaaagccaagaagtcggtggacaagaacagcaacgagtaccgggtacggcgggaacgcaacaacatcgcggtgcgcaagagccgagataaagccaaacaacgcaacgtggagacgcaacagaaggtgctggagttgaccagtgacaatgaccgcctgcgcaagcgggtggaacagctgagccgtgaactggacacgctgcggggcatcttccgccagctgcctgagagctccttggtcaaggccatggcaactgcgcgtgaggcgcgcggctgcgggaccgccttgggccggccccctggctggagacccagaggatggtttcgggtcgctggatctctaggctgcccgggccgcgcaagccaggactaggagattccggtgtggcctgaaagcctggcctgctccgcgtgtcccctcccttcctctgagccggactcggtgcgtctaagatgagggagtcaggccgtggtggtttctccttgagaccgagagacttttccgcggagctgagctgggggcccggcagtactagtaagcttaataaaaacagacaataaaagaattggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcgatctttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcaggactcttcctttttcaataattctctagcactttgtagaccaggctggcctcgaactcagaaatccgcctgcctctgcctcccgagtgctgggattaaaggtgtgcgccaccacacccggctctccaggtatcttgacttaagcattctatatactgttgaagaccaggtatagatctaagtggtagatagatttagatgtaatgtaattttatgtaagaggcttataaaataacagcagtgttcaacacaatcaattttggatgtgttccatgctcaatttttttttgatattaattatttagtagggtctcatgtctagttcttcgggttgacagaaataacattttcactctcaatctttttaaaagtgctaattgttcgactctctggaaagcctaatgtgaacaggccgactgtagcataattatgtccaaagctactaaatgaagccattttgaatattatactgtgagggagccaagtagtaaggggacctgaaagatgagtattagtactaagtctgcaactgatcattctggttaactttgttccttaatttttcagtgtccccaagtgtaaaattaagatgtctggacaggtggttcctaacaacttggcagttctaagacttcatggcttgggtttaaattccacaacgattgctttcccggttaaaatcctcacttgtccaagtctgttttcctttcagcttccacgttagattcctcccagtgcgtgacggagaagaatgtgcttagcatgtaagtttcgcttccatcaatgtgctgcagaaatggcgtgcagagataagtctaggcaaccgaatgtgaggtttgaaattacaggagcaaattgggcagctttaaggctttgcttgtgctgaatatcacactgataagcaacatgtttaacttcggtcaacgctttagatcgagtcgactccctttagtgagggttaattgctagagcggccgaccgtagagtcgagcagtgtggttttcaagaggaagcaaaaagcctctccacccaggcctggaatgtttccacccaatgtcgagcagtgtggttttgcaagaggaagcaaaaagcctctccacccaggcctggaatgtttccacccaatgtcgagcaaaccccgcccagcgtcttgtcattggcgaattggaacacgcagatgcagtcggggcggcgcggtcccaggtccacttcgcatattaaggtgacgcgtgtggcctcgaacaccgagcgaccctgcagcgacccgcttaacagcgtcaacagcgtgccgcacatcttggtggcgtgaaactcccgcacctcttcggccagcgccttgtagaagcgcgtatggcttcgtaccccggccatcagcacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgccttgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccttcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatgatccagacccacgtcaccaccccaggctccataccgacgatctgcgacctggcgcgcacgtttgcacgggagatgggggaggctaactgaaacacggaaggagacaataccggaaggaacctgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggcctgccatagccacgggccccgtgggttagggacggggtcccccatggggaatggtttatggttcgtgggggttattattttgggcgttgcgtggggtcagtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgccgccggacgaactaaacctgactacggacggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggccccccctcgaggtcgacggtatcgataagcttgatatcg

SEQ ID NO: 3 - DNA, 22 bp, Artificial
  Oligonucleotide Primer
tggacaagaacagcaacgagta

SEQ ID NO: 4 - DNA, 19 bp, Artificial
  Oligonucleotide Primer
gcagttgccatggccttga

SEQ ID NO: 5 - DNA, 22 bp, Artificial
  Oligonucleotide Primer
ccatgaacagcaagggttgtaa

SEQ ID NO: 6 - DNA, 20 bp, Artificial
  Oligonucleotide Primer
tggaagtgggcaaccacata

(19) United States
(12) Patent Application Publication
(10) Pub. No.: US 2007/0072296 A1
(43) Pub. Date: Mar. 29, 2007

(54) C/EBPalpha gene targeting constructs and uses thereof

(75) Inventor: Nai-dy Wang, Singapore (SG)

Correspondence Address: ROTHWELL, FIGG, ERNST & MANBECK, P.C., 1425 K STREET, N.W., SUITE 800, WASHINGTON, DC, 20005, US

(73) Assignee: National University of Singapore, Singapore (SG)

(21) Appl. No.: 11/514,903

(22) Filed: Sep. 5, 2006

Related U.S. Application Data
(63) Continuation of application No. 10/703,224, filed on Nov. 6, 2003, now abandoned.

(51) Int. Cl. C12N 15/09; C07H 21/04; C12N 5/08

(52) U.S. Cl. 435/455; 435/325; 435/370; 435/320.1; 536/23.2

(57) ABSTRACT

A DNA construct for the expression of a functional mammalian C/EBPα comprising the following components: (i) a first region having homology with an endogenous tumor-specific promoter gene sequence of a gene locus (ii) a DNA molecule encoding the C/EBPα; and (iii) a second region having homology with a region within the gene locus. The DNA construct is used for the preparation of non-human knock-in animals.

### FIELD OF THE INVENTION

The present invention relates to DNA constructs encoding C/EBPα and to the use of said constructs for the inhibition or prevention of tumors. The invention further relates to the preparation of knock-in non-human animals and hepatocytes expressing exogenous C/EBPα under the control of a tumor-specific promoter.

### BACKGROUND OF THE INVENTION

The murine CCAAT/enhancer binding protein α (C/EBPα) is a 395 amino acid transcription factor encoded by a single-exon gene (Christy, R. J., et al., 1991, Proc. Natl. Acad. Sci. USA 88:2593-2597). This heat-stable transactivator is highly expressed in hepatic and adipose tissues but is detectable at lower levels in the brain, gut, and lungs (Birkenmeier, E. H., et al., 1989, Genes Dev. 3:1146-1146; Friedman, A. D., et al., 1989, Genes Dev. 3:1314-1322; Johnson, P. F., et al., 1987, Genes Dev. 1:133-146). A member of the bZIP family of proteins, C/EBPα recognizes and binds to specific DNA sequences through a basic DNA-binding domain and a leucine zipper dimerization domain. A peptide sequence of twenty-four predominantly basic amino acids serves as the DNA contact region. Immediately adjacent to this region is a thirty-five residue a helical coil containing four leucines each spaced seven amino acids apart. These leucines align on the same side of the α helix and interdigitate with other proteins carrying the same leucine motif to “zip” the proteins together. C/EBPα can form both homodimers as well as heterodimers. The consensus binding site for C/EBPα homodimers is T(T/G)NNG(C/T)AA(G/T) although tests in vitro indicate that highly divergent sequences also can be bound (Vinson, C. R., et al., 1989, Science 246:911-916; McKnight, S. L., et al., 1989, Genes Dev. 3:2021-2024).

Two important functions had long been ascribed to C/EBPα based on numerous in vitro studies. The first is that of a transcriptional regulator that induces cellular growth arrest and terminal differentiation in hepatocytes and adipocytes (Freytag, S. O. and Geddes, T. J., 1992, Science 256:379-382; Mischoulon, D., et al., 1992, Mol. Cell. Biol. 12:2553-2560). The second is that of a regulator that transactivates energy-related genes to establish and maintain energy homeostasis (Kaestner, K. H., et al., 1990, Proc. Natl. Acad. Sci. USA 87:251-255; Park, E. A., et al., 1990, Mol. Cell. Biol. 10:6264-6272; McKnight, S. L., et al., 1989, Genes Dev. 3:2021-2024).

Early investigations into the functions of C/EBPα had identified an inverse correlation between its expression and cellular proliferation. Work in 3T3-L1 adipoblasts demonstrated that premature expression of C/EBPα could induce cellular growth arrest and terminal differentiation (Umek, R. M., et al., 1991, Science 251:288-292; Freytag, S. O. and Geddes, T. J., 1992, Science 256:379-382). Observations in rat primary hepatocytes and partial hepatectomies revealed a greater than 80% drop in C/EBPα transcripts as the cells progressed into the growth phase (Mischoulon, D., et al., 1992, Mol. Cell. Biol. 12:2553-2560.). Western analyses showed cultured hepatoma cells to contain approximately 5% of the amount of C/EBPα as the normal rat liver (Friedman, A. D., et al., 1989, Genes Dev. 3:1314-1322). Proliferation of hepatoma cells and transformed hepatocytes is inhibited by transfection of constitutively expressing C/EBPα genes (Hendricks-Taylor, L. R. and Darlington, G. J.,1995, Nucleic Acids Res. 23:4726-4733; Diehl, A. M., et al., 1996, J. Bio. Chem. 271:7343-7350).

Other in vitro experiments showed C/EBPα to transactivate the promoters of many energy-related genes including the following: albumin which binds and transports long chain fatty acids in serum, the lipid-binding protein (422/aP2) which binds and transports long chain fatty acids in adipocytes, stearoyl acyl-CoA desaturase 1 (SCD1) which desaturates fatty acids for transport and storage in adipocytes, the insulin-responsive glucose transporter (GLUT4) which controls entry of glucose into adipocytes for lipogenesis, and phosphoenolpyruvate carboxykinase (PEPCK) which is a key enzyme involved in gluconeogenesis (Kaestner, K. H., et al., 1990, Proc. Natl. Acad. Sci. USA 87:251-255; Park, E. A., et al., 1990, Mol. Cell. Biol. 10:6264-6272). The ability of C/EBPα to transactivate these genes led to the proposal that its primary function is that of a regulator of energy metabolism (McKnight, S. L., et al., 1989, Genes Dev. 3:2021-2024).

The principal organ involved in the maintenance of energy homeostasis in mammals is the liver. Hepatic C/EBPα mRNA is detectable on the thirteenth day of the twenty-one day mouse gestation period; it rises by day 17 and reaches a maximum near the time of birth (Birkenmeier, E. H., et al., 1989, Genes Dev. 3:1146-1146; Kuo, C. F., et al., 1990, Dev. 109:473-481). High C/EBPα expression during the prenatal term coincides with the initiation of hepatic glycogen storage.

Rodents, in a time frame analogous to humans, gain the capacity to synthesize and store large amounts of hepatic glycogen during the last trimester of fetal life, setting the stage for the shift from fetal metabolic processes to those of the neonate (Jones, C. T., 1982, The Development of the Metabolism in the Fetal Liver. New York: Elsevier Biomedical Press). Mobilization of this stored glycogen at birth acts to maintain blood glucose homeostasis. Gluconeogenesis begins within hours thereafter. Attendant with these metabolic changes is the up-regulation in expression of the energy-related liver enzymes, PEPCK, tyrosine aminotransferase (TAT), acetyl-CoA carboxylase, albumin, glucose-6-phosphatase (G6Pase), and glycogen synthase (Park, E. A., et al., 1992, J. Biol. Chem. 267:613-619; Grange, T., et al., 1991, Nucleic Acids Res. 19:131-139.; Tea, H. J., et al., 1994, J. Biol. Chem. 269:10475-10484; Wang N. D., et al., 1995, Science 269:1108-1112). Members of the C/EBP family of transcription factors have been implicated in the transactivation of these genes.

U.S. Pat. No. 5,545,563 ('563) discloses a vector for transient transfection in mammals expressing C/EBPα under the control of exogenous CMV or other exogenous tumor-specific promoter. '563 further discloses a replacement or insertion vector, capable of expressing a mutant C/EBPα, for the preparation of knock-out (or deletion) animals, which possess predetermined mutations and alleles in their chromosomal C/EBPα genes. According to this method, the naturally present gene sequence of a cell of the animal is replaced with an analog sequence capable of causing the expression of an analog C/EBPα that is a sequence which has been altered compared to the naturally occurring C/EBPα. The hepatocytes of the knock-out animal prepared with the method disclosed in U.S. Pat. No. 5,545,563 are prone to immortalization and accordingly the animals are predisposed to cancer.

Other knock-out mice have been disclosed in the art (Wang, N. D., et al., 1995, Science 269:1108-1112) and used for the analysis of the role of C/EBPα in promoting tissue maturation as well as in the production and maintenance of life-sustaining fuel levels in the neonate. The absence of C/EBPα in these mice gave rise to conditions that greatly resemble the symptoms of the human premature infant including growth retardation, severe hypoglycemia, insufficient hepatic glycogen storage and insufficient adipose fat storage.

The problem related to the knock-out animals disclosed in the prior art is that these knock-out animals have abnormal liver function, experience severe hypoglycaemia, and die within a few hours post-partum. Furthermore, their hepatocytes have been shown to be prone to tumourigenesis (Soriano, H. E., et al., 1998, Hepatology 27:392-401). Accordingly, these knock-out animals are not useful for producing, or for testing, potential therapeutic applications of C/EBPα-expressing cells and tissues. Another problem associated with the U.S. Pat. No. 5,545,563 disclosure is its suggestion to express C/EBPα under the regulation of the α-fetoprotein (AFP) promoter in a mammalian vector.

Because the normal regulation of AFP is complex and its complete array of regulatory sequences may not be known for some time to come, expression systems that rely on the use of cloned AFP promoter DNA run the risk of rendering inaccurate AFP regulation. Unintended gene expression is a risk inherent in all current gene therapy strategies.

It would therefore be desirable to identify and provide new and different means for the generation of tumor-resistant cells and animals which could be useful in the treatment of tumors.

### SUMMARY OF THE INVENTION

The present invention seeks to address the problems above, and in particular to provide new and useful means for the generation of tumor-resistant cells and animals.

One aspect of the invention is a DNA construct for the expression of a functional C/EBPα, comprising a DNA molecule encoding C/EBPα.

The DNA construct according to the invention preferably comprises, in a 5′ to 3′ end direction reading frame, the following components:

(i) a first region having homology with an endogenous tumor-specific promoter gene sequence of a gene locus;

(ii) a DNA molecule encoding the C/EBPα; and

(iii) a second region having homology with a region within the gene locus.

The DNA molecule encoding the C/EBPα and a second region of homology is positioned downstream of the tumor-specific promoter sequence.

Such a construct allows the expression of the exogenous C/EBPα under the control of the endogenous tumor-resistant promoter of a host cell or animal. The DNA construct according to the invention allows the expression of C/EBPα derived from any organism, preferably a mammal, such as human or mouse C/EBPα.

Preferably, the first region has homology with an endogenous tumor-specific promoter of a gene locus, which is activated in liver and lung tumors. More preferably, the first region is homologous with an endogenous mammalian α-fetoprotein (AFP) promoter of the AFP gene locus and the second region is homologous with a region within the AFP gene locus. The second region being downstream of the AFP promoter.

Accordingly, an embodiment of the invention is a DNA construct for expression of a functional mammalian C/EBPα comprising, in a 5′ to 3′ direction, the following components:

(i) a first region having homology with an endogenous AFP promoter gene sequence of the AFP locus;

(ii) a DNA molecule encoding the C/EBPα gene; and

(iii) a second region having homology with a region within the endogenous AFP gene locus.

The first and second regions of homology with the endogenous tumor-specific promoter (for example AFP promoter) of the AFP locus and within the tumor-specific promotergene locus are also indicated as “upstream” and “downstream” sequence, respectively, as they are positioned, in the DNA construct, upstream and downstream to the DNA molecule encoding the exogenous C/EBPα of interest. The upstream and downstream sequences allow the positioning of the DNA molecule encoding the exogenous C/EBPα of interest into the endogenous tumor-specific promoter locus (for example, into the AFP locus).

The invention also provides an expression vector into which said DNA construct is inserted.

Another aspect of the invention relates to host cells comprising the DNA construct or the expression vector. The host cell can be a human or animal cell, for example a human or animal hepatocyteorpneumocyte. The human or animal cell in which the DNA construct is inserted becomes a tumor-resistant cell. Accordingly, the present invention provides tumor-resistant cells, preferably hepatocytes or pneumocytes, which can be used in therapy. For example, tumor-resistant hepatocytes, hepatocyte aggregates or hepatocyte grafts according to the invention can be administered to an area in need of transplant in a mammal, such as a human or animal.

The tumor-resistant cells can be prepared by transfecting host cells with the DNA construct or the expression vector according to the invention. These tumor-resistant cells can also be isolated from knock-in animals which have been created using the DNA construct of the invention.

The tumor-resistant cells, according to the invention, can also be human or animal pluripotent embryonic or adult stem cells transfected with the DNA construct of the invention. Preferably, such transfected pluripotent embryonic or adult stem cells are cultivated and differentiated into tumor-resistant hepatocytes or pneumocytes.

The invention provides a process for preparing a hepatocyte graft for administration to an area in need of transplant, wherein the process comprises making the hepatocytes of the graft tumor-resistant by transfecting these hepatocytes with the DNA construct.

Also provided is a method for transplanting hepatocytes, hepatocyte aggregates or hepatocyte grafts, comprising preparing hepatocytes, hepatocyte aggregates or hepatocyte grafts transfected with the DNA construct, and transplanting the prepared transfected hepatocytes, hepatocyte aggregates or hepatocyte grafts to an area in need of transplant.

The invention also relates to bioreactors, preferably, bioartificial livers, comprising the use of the tumor-resistant cells according to the invention. A further aspect of the invention is a knock-in non-human animal transfected or modified with the DNA construct according to the invention. In such a knock-in non-human animal, the exogenous C/EBPα transgene is specifically transcribed by the animal's endogenous tumor-specific promoter. Preferably, the endogenous tumor-specific promoter is the AFP promoter, which transcribes the exogenous C/EBPα transgene specifically in the fetal liver at a point earlier than normal or in developing cancer cells where the AFP promoter is reactivated.

The invention provides a method for the preparation of knock-in non-human animals modified with the DNA construct of the invention. For example, pluripotent non-human animal embryonic stem cells modified by the DNA construct of the invention could be used in blastocyst microinjections to produce chimeric animals that can propagate the modified allele. Alternatively, somatic cells of an animal could be modified in culture by transfection with the DNA construct of the invention and used as donors for nuclear transfer to generate viable animals carrying the modified allele.

Also provided are tumor-resistant cells isolated from these knock-in animals. Such tumor-resistant cells are preferably tumor-resistant hepatocytes or pneumocytes isolated from these knock-in animals.

These tumor-resistant hepatocytes can be used to populate bioartificial livers that provide essential liver function for patients with liver failure.

The present invention also provides a method for in vivo screening and/or modulation of tumors comprising administering one or more carcinogens to a non-human animal transfected with the DNA construct of the invention and identifying the tumors which are inhibited or suppressed by the expression of transgenic C/EBPα.

A further aspect of the invention is a method for treating symptoms associated with liver glycogen-insufficiency in a human premature infant or a premature animal comprising administering into, injecting into or transfecting transiently the liver of the human premature infant or the premature animal with the circular (non-linearized) form of the DNA construct of the invention. The circularform of the construct is less likely to integrate into the host genome. Transient transfection of the host cell is desirable because the need for artificial C/EBPα expression is only necessary until the host's own endogenous C/EBPα expression becomes activated.

A still further aspect is a method of treating cancer in human or animals whose cells produce elevated levels of AFP comprising treating the human or animal cancer with the circular form of the DNA construct of the invention.

The invention also provides a pharmaceutical preparation comprising the construct of the invention and, optionally, a pharmaceutically acceptable carrier and/or diluent .

Further, the invention relates to the use of a DNA construct according to the invention for the manufacturing of a pharmaceutical preparation for use in medicine. For example, for the treatment of symptoms associated with glycogen-insufficiency in a human premature infant or premature animal; and for the treatment of cancer in humans or animals whose cells produce elevated levels of AFP (treating the human or animal cancer with the circular form of the DNA construct of the invention).

### BRIEF DESCRIPTION OF THE FIGURES

FIG. 1 is an illustration of the AFP-C/EBPα gene targeting construct. The C/EBPα transgene is striped. The stippled regions represent the homologous sequences that help mediate homologous recombination. The ID tag is an artificially inserted sequence containing a HinDIII restriction enzyme site; it assists in differentiating transcripts produced by the transgene from endogenous C/EBPα mRNA.

FIG. 2 shows a restriction enzyme map of the AFP-C/EBPα gene targeting plasmid. *—indicates approximate location of restriction enzyme site.

FIG. 3A-C is the sequence of the AFP-C/EBPα gene targeting plasmid. The letter “N” denotes undetermined sequence. Sequence data for the AFP upstream and downstream sequences was obtained from GenBank NCBI (National Center for Biotechnology Information) to synthesize PCR probes used to screen a 129SvJ murine genomic lambda-phage library purchased from Stratagene. Nucleotides 1-5548 comprises the upstream promoter and regulatory sequences of AFP (also shown in SEQ ID NO:1). Nucleotides 8707-9612 comprises the AFP downstream gene sequence starting from the inside of intron 1 up to part of intron 3 (also shown in SEQ ID NO:2). The section of nucleotides 757-4285 represents the subsection of the AFP gene that has not been sequenced.

FIG. 4 is a schematic of the process used to create the C/EBPα gene knock-in mouse.

FIG. 5 is a restriction enzyme map of the wildtype C/EBPα allele and the knock-in allele. The 11.0 kb fragment in PstI digested DNA and a 3.7 kb fragment in HindIII digested DNA indicate the presence of the knock-in allele. The 3.4 kb PstI fragment and 8.0 kb HindIII fragment show the presence of the endogenous C/EBPα gene.

FIG. 6 shows Southern blots revealing the genotypes of the knock-in (KI) mice versus that of wildtype (WT) mice using PstI digested DNA (Panel A) and HindIII digested DNA (Panel B).

FIG. 7 shows a graph of the expression pattern of the knock-in C/EBPα (KI C/EBPα) gene as compared to normal C/EBPα expression.

FIG. 8 shows a graph of real-time PCR data illustrating the expression profiles of C/EBPα and glycogen synthase (GS) in knock-in (KI) and wildtype mice. The data shows early and higher expression of KI C/EBPα as well as higher expression of KI GS in comparison to wildtype mice.

FIG. 9 shows that mRNA levels of C/EBPα were higher in the livers of knock-in versus wildtype mice between 12.5 and 15.5 days of gestational age.

FIG. 10 shows that mRNA levels of glycogen synthase were higher in the livers of knock-in versus wildtype mice between 12.5 and 15.5 days of gestational age.

FIG. 11(A, B, C, D) shows liver sections of mice at 15.5 days gestational age. Slides treated with or without Diastase were stained with PAS. A) KI PAS—the pink hue (darker cells) shows the presence of hepatic glycogen in the knock-in liver, B) KI PAS Diastase—diastase has cleared the slide of glycogen, C) WT PAS—wildtype animals have not yet deposited glycogen at this time point, D) WT PAS Diastase

FIG. 12 shows cultures of fetal primary hepatocytes derived from knock-in mice were more likely to die in response to exposure to genotoxic agents than cells from wildtype mice.

FIG. 13 shows knock-in and wildtype cells cultured in the absence of genotoxic agents have similar survival rates.

FIG. 14(A, B, C, D) shows livers from carcinogen treated wildtype mice exhibit pronounced tumor formation (A and B), and livers from carcinogen treated knock-in mice (C and D) exhibit smaller and fewer tumor growths than the wildtype mice.

### DETAILED DESCRIPTION OF THE INVENTION

One aspect of the present invention relates to novel constructs that can be used to transform host cells so that they express and secrete (i.e., into the host cell environment or into the knock-in organism) exogenous C/EBPα. Preferably, the DNA construct according to the invention is a DNA construct for the expression of a functional C/EBPα comprising, in a 5′ to 3′ end direction reading frame, the following components:

(i) a first region having homology with an endogenous tumor-specific promoter gene sequence of a gene locus;

(ii) a DNA molecule encoding C/EBPα; and

(iii) a second region having homology with a region within the gene locus.

The DNA molecule encoding the C/EBPα and a second region of homology is positioned downstream of the tumor-specific promoter sequence.

Such a DNA construct allows the expression of the exogenous C/EBPα under the control of the endogenous tumor-specific promoter.

The DNA construct according to the invention allows the expression of C/EBPα derived from any organism, preferably mammal, for example human or mouse C/EBPα.

The construct of the present invention produces results that are particularly advantageous over the construct suggested in the prior art. For example, the vector suggested by '563 which relies on expression from a cloned exogenous promoter has the problem of lacking the complete set of regulatory elements leading to imprecise gene expression.

The DNA molecule can be any gene molecule from any organism encoding any C/EBPα known in the art or any derivative or mutation thereof.

Examples of known C/EBPα genes are for example those disclosed in the NCBI UniGene web page (http://www.ncbi.nlm.nih.gov/). For example, but not limited to, C/EBPα genes of Mus musculus, UniGene cluster Mm,34537; Homo sapiens, UniGene cluster Hs.76171; Rattus norvegicus, Unigene cluster Rn.22163 (all of them herein incorporated by reference).

The human C/EBPα gene has also been disclosed in U.S. Pat. No. 5,545,563 (herein incorporated by reference).

Sequence data for the AFP upstream and downstream sequences was obtained from GenBank NCBI (National Center for Biotechnology Information) to synthesize PCR probes used tb screen a 129SvJ murine genomic lambda-phage library purchased from Stratagene.

Accordingly, the DNA construct according to the invention comprises a DNA molecule encoding C/EBPα of any organism, preferably, mammal or more preferably, human or animal, for example mouse, C/EBPα.

The C/EBPα of the present DNA construct is preferably a naturally functional C/EBPα. However, any functional C/EBPα can be used. It is within the knowledge of a skilled person in the art how to prepare any functional C/EBPα analog or mutant obtained by a deletion, insertion and/or substitution of at least one nucleotide of the naturally occurring C/EBPα.

The C/EBPα gene used in the preparation of the DNA construct can be isolated according to a variety of standard techniques, for example, isolated from human or animal cDNA libraries. Human or animal cDNA libraries can be obtained from any human or animal cell, for example, from a hepatoma or other cell that actively expresses C/EBPα. The DNA molecule may be cloned into any suitable prokaryotic or eukaryotic vector and the vectors then are screened using a probe whose sequence is the same as, or complementary to, that of the desired C/EBPα or to a fragment thereof.

Suitable vectors, as well as the Methods for performing such hybridization screenings are described, for example, in Sambrook, J. and Russel D. W., In: Molecular Cloning A Laboratory Manual, 3nd Edition, (Cold Spring Harbor Laboratory Press, Cold Spring Harbor, N.Y. (2001), herein incorporated by reference). Alternatively, amplification procedures, such as PCR are used to facilitate the isolation of the C/EBPα-encoding DNA. PCR methods are reviewed by Love, S. et al., 1992, Neuropath. Appl. Neurobiol. 18:95-111.

According to another alternative, synthetic chemical methods may be used to synthesize the C/EBPα gene molecule. Thus, for example, the well-known phosphodiester or phosphotriester methods may be used.

The first region of the DNA construct of the invention has homology with the sequence of an endogenous tumor-specific promoter of a gene locus. The second region of the DNA construct is a region having homology with a region within the tumor-specific promoter gene locus that lies downstream of the tumor-specific promoter. Within the DNA construct, this second region of the DNA construct lies downstream of the C/EBPα gene.

Usually, the promoter locus would include all of the gene regulatory elements upstream (5′) of the coding sequence. For the second region of homology (the region within the gene locus), it is convenient to use a homologous sequence that is at least 500 base-pairs long or longer. The purpose of the two homologous regions is to allow DNA cross-over events to insert construct sequences into the genome. The greater the length of homology, the more efficient gene targeting should be. For the invention's construct, particular care was taken to retain the portion of AFP's intron 1 sequence believed to contain additional AFP gene regulatory elements.

The first and second regions can be chosen to have homology to suitable endogenous mammalian promoters (i.e. promoters that are more active in tumor cells than in non-tumor cells). Examples of such a promoter include, the α-fetoprotein (AFP) promoter, the amylase promoter, the cathepsin E promoter, the M1 muscarinic receptor promoter, the gamma-glutamyl transferase promoter, the CMV promoter or the like. Preferably, the first and second regions of the DNA construct have homology with the AFP promoter and a region within the AFP locus.

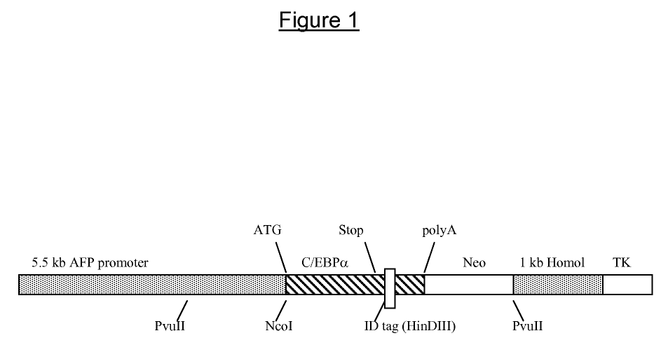

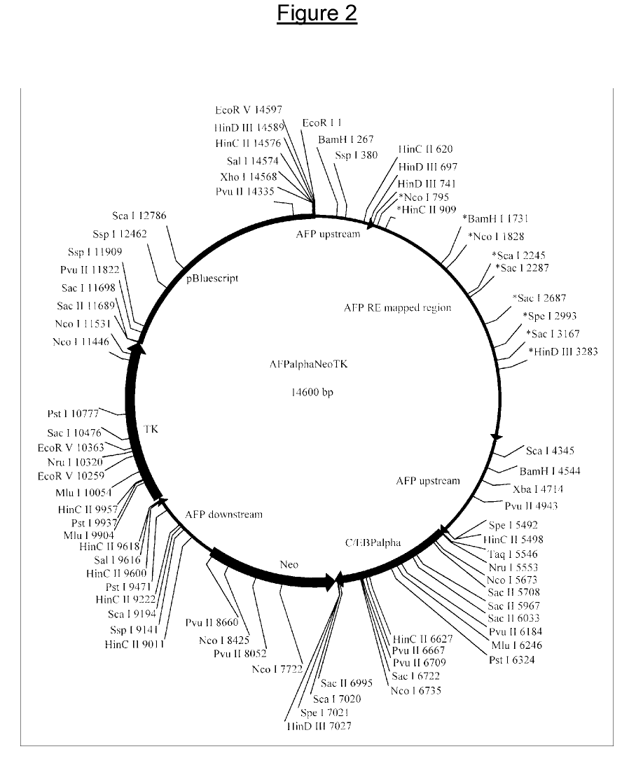

The first and second regions of homology with the endogenous tumor-specific gene (for example AFP) will preferably flank the DNA molecule encoding C/EBPα. These first and second regions are also indicated as “upstream” and “downstream” sequences, respectively, as they are positioned, in the DNA construct, upstream and downstream to the DNA molecule encoding the exogenous C/EBPα of interest. The upstream and downstream sequences allow the positioning of the DNA molecule encoding the exogenous C/EBPα of interest into the endogenous tumor-specific locus (for example, into the AFP locus) and in particular to be under the control of the endogenous tumor-specific promoter (ex. AFP promoter). The positions of the upstream and downstream regions and the DNA molecule encoding C/EBP are shown in FIG. 1.

Suitable C/EBPα and AFP sequences can be prepared from Stratagene's Mouse Lambda Genomic Library—129SvJ (Catalog #946313).

The fetal oncoprotein, α-fetoprotein (AFP), is synthesized primarily in the embryonic liver and yolk sac (Seliem, C. H., et al., 1984, Dev. Biol. 102:51-60; Dziadek, M. A. and Andrews, G. K., 1983, EMBO J. 2:549-554). AFP messenger RNA is detectable in the hepatic primordia which form on the 10th day of the 21-day mouse gestation period (Dziadek, M. A. and Andrews, G. K., 1983, EMBO J. 2:549-554; Kaufman, M. H., 1992, The Atlas of Mouse Development. London: Academic Press). Expression peaks in the fetal liver by the 19th day and decreases to negligible amounts after birth (Sellem, C. H., et al., 1984, Dev. Biol. 102:51-60). The AFP prenatal expression pattern precedes fetal C/EBPα expression in the liver by 2-3 days where C/EBPα mRNA is detectable on day 13, peaks near the time of birth, and then also drops after birth (Birkenmeier, E. H., et al., 1989, Genes Dev. 3:1146-1146; Kuo, C. F., et al., 1990, Dev. 109:473-481).

AFP transcription normally remains off in the adult; in contrast, C/EBPα transcription rises to a maximum again as the animal enters the adult stage of development at six weeks of age (Darlington, G. J., et al., 1995, Curr. Opinion Genet. Dev. 5:565-570). The increased production of C/EBPα in adulthood is consistent with the decreased rate of hepatocyte proliferation at this stage. Interestingly, during periods of coerced hepatic cell division such as in partial hepatectomies and tumorigenesis, C/EBPα transcription becomes suppressed while AFP expression becomes reactivated (Mischoulon, D., et al., 1992, Mol. Cell. Biol. 12:2553-2560; Tomizawa, M., et al., 1998, Biochem. Biophys. Res. Comm. 249:1-5).

This inverse relationship in gene activation between C/EBPα and AFP represents an ideal condition for artificially expressing C/EBPα under the regulatory elements of the AFP gene in order to enlist C/EBPα as an inhibitor of tumor growth during chemically induced carcinogenesis.

According to a preferred embodiment, the first and second regions of the DNA construct of the invention have homology with the endogenous human or animal AFP promoter of a gene locus and with a downstream sequence region within the gene locus. Accordingly, with standard gene targeting techniques, a single-copy of the exogenous C/EBPα transgene is placed under the control of the endogenous human or animal tumor-specific promoter (ex. AFP promoter) to create a cell or a knock-in non-human animal capable of producing exogenous C/EBPα at a time different from the endogenous C/EBPα. For example, when the exogenous C/EBPα transgene is placed under the control of the endogenous AFP gene promoter to create, for example, a C/EBPα gene knock-in mouse strain, the exogenous C/EBPα is transcribed specifically in the fetal liver at a time point earlier than normal as well as in developing liver tumor cells.

The DNA construct of the invention preferably further comprises at least one selectable marker gene sequence.

As dominant gene sequence, the neomycin gene selectable marker can be used for positive selection (for instance PGKneo). The neomycin resistance gene confers a survival advantage to a cell cultured in the presence of neomycin or G418.

Other examples of selectable marker gene sequences include the hprt gene, the tk gene (preferably the MC1tk cassette, that is, the Herpes Simplex Virus type 1 thymidine kinase), the nptII gene (Thomas, K. R. et al., 1987, Cell 51:503-512; Mansour, S. L. et al., 1988, Nature 336:348-352, both herein incorporated by reference), or other genes which confer resistance to amino acid or nucleoside analogs, or antibiotics, etc. The selectable or detectable marker gene may be any gene which can complement for a recognizable cellular deficiency. Most preferably, the DNA construct may contain flanking gene sequences capable of “negative selection” as well as “positive selection,” such as the tk gene that encodes thymidine kinase, or the hprt gene that encodes hypoxanthine phosphoribosyl transferase. Cells expressing active thymidine kinase are able to grow in media containing HATG, but are unable to grow in media containing nucleoside analogs such as 5-azacytidine (Giphart-Gassler, M. et al., 1989, Mutat. Res. 214:223-232). Cells containing an active HSV-tk gene are incapable of growing in the presence of gancyclovir or similar agents (Giphart-Gassler, M. et al., 1989, Mutat. Res. 214:223-232). Cells which express an active HPRT enzyme are unable to grow in the presence of certain nucleoside analogs (such as 6-thioguanine, 8-azapurine, and the like), but are able to grow in media supplemented with HAT (hypoxanthine, aminopterin, and thymidine). Conversely, cells which fail to express an active HPRT enzyme are unable to grow in media containing HATG, but are resistant to analogs such as 6-thioguanine, and the like (Littlefield, J. W., 1964, Science 145:709-710).

The DNA construct according to the invention can be introduced into a cell, for example a hepatocyte, a proliferating somatic cell, a pluripotent stem cell or an adult stem cell, by any method which allows the introduced molecule to undergo recombination at its regions of homology. Examples of such standard methods are electroporation, microinjection, calcium phosphate transformation and the like.

After transformation, the cells are cultured in medium that selects for transfectants that have received and integrated the positive selectable marker gene sequence. The culturing conditions are then altered, such that they can select against recipient cells that have acquired the negative gene. A positive and negative selection may also be accomplished simultaneously. Since all of these sequences are present on the same targeting vector molecule, such dual selection requires a recombinational event to occur.

The survivors of the selection are then screened either for the function of the chromosomal allele, or more preferably, by Southern blot analysis for clones in which the selectable marker gene sequence has been introduced into the chromosomal allele.

After selection for cells which have incorporated the desired DNA molecule (for example by selection for G418 resistant cell), the cells are cultured, and the presence of the introduced DNA molecule is confirmed as described above. Any of a variety of methods may be used to identify cells which have undergone the desired recombinational event. Direct screening of clones, use of PCR, use of hybridization probes, etc., may all be employed for this purpose.

The DNA construct of the invention may also comprise an identification (ID) tag, which is used to differentiate transcripts produced by the transgenic C/EBPα from the endogenous C/EBPα mRNA, for example, by introducing a novel restriction enzyme site into the sequence, the transgenic C/EBPα cDNA can be distinguished from the endogenous gene product through the use of PCR and/or restriction enzyme digestion. The positioning of an ID tag incorporating a novel HinDIII site in one embodiment of the DNA construct of the invention is shown in FIG. 1.

The DNA construct of the invention may be incorporated into vector molecules. The term vector, as used herein is intended to denote any viral or plasmid molecule that is capable of being introduced (as by transformation, electroporation, transfection, or the like) and/or propagated (i.e. replicated) in a prokaryotic or eukaryotic cell.

Preferred prokaryotic vectors include plasmids such as those capable of replication in E. coli such as, for example, pBR322, ColE1, pSC101, pACYC 184, piVX. Such plasmids are, for example, disclosed by Sambrook, J. and Russel D. W., 2001, In: Molecular Cloning, A Laboratory Manual, 3^{rd} Ed., Cold Spring Harbor Press, Cold Spring Harbor, N.Y. Bacillus plasmids include pC194, pC221, pTl27, and the like. If desired, yeast and fungal vectors may be used. Examples of suitable yeast vectors include the yeast 2-micron circle, the expression plasmids YEP13, YCP and YRP, etc., or their derivatives. Such plasmids are well known in the art (Botstein, D., et al., 1982, Miami Wntr. Symp. 19:265-274). Examples of mammalian vectors are disclosed by Sambrook, J. and Russel D. W., 2001, as above.

The C/EBPα-encoding sequences may be expressed in mammalian cells, using eukaryotic vectors (especially, a eukaryotic viral or retroviral vector). Typically, such vectors are designed to include a prokaryotic replicon, and selectable marker, such that the propagation of the vector in bacterial cells can be readily accomplished. In order to replicate in mammalian cells, however, the vectors will also contain a viral replicon, such as the replicon of Epstein-Barr virus, bovine papilloma virus, parvovirus, adenovirus, or papovirus (i.e. SV40 or polyoma virus).

Accordingly, the invention relates to any kind of expression vector comprising the DNA construct of the invention.

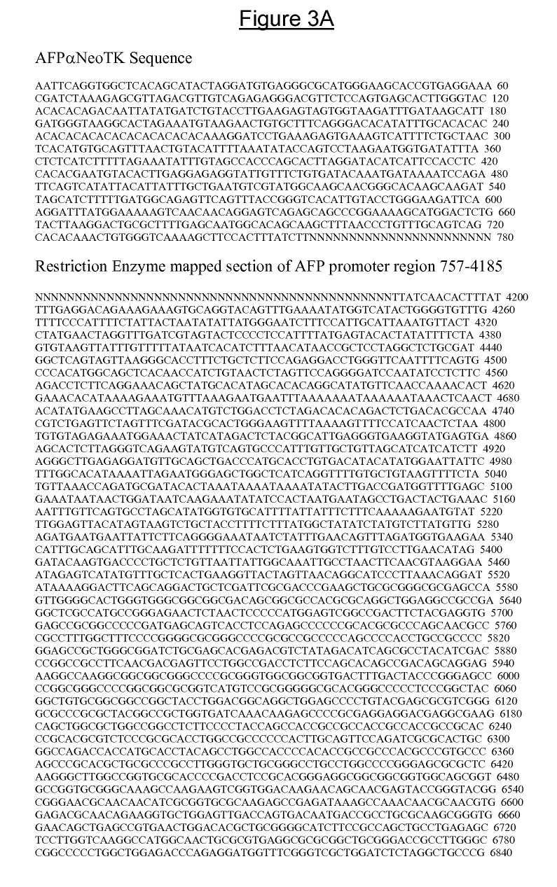

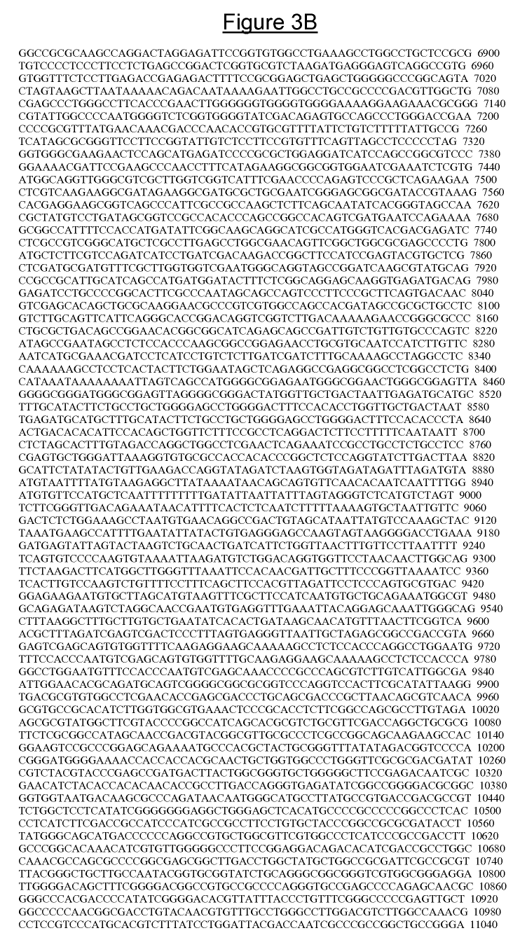

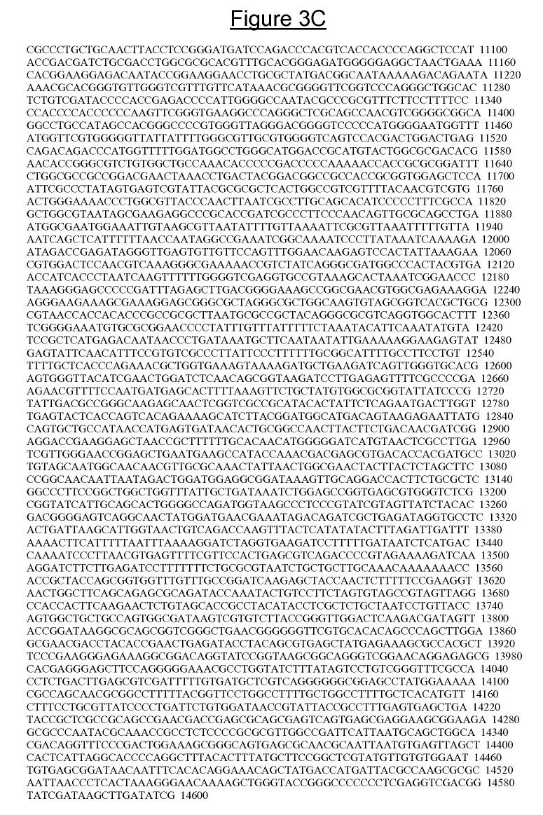

An example of an expression vector is the mouse AFP-C/EBPα gene targeting plasmid disclosed in FIG. 2 and FIG. 3A-C. The expression vector according to the invention may comprise or consist of the plasmid construct of FIG. 2 and FIG. 3A-C. Essential portions of the nucleotide sequence of the plasmid APFαNeoTK are shown in SEQ ID NO:1 and SEQ ID NO:2.

The DNA construct or expression vector according to the invention allows the positioning of the exogenous C/EBPα inside the chromosome of the host cell, under the control of the host cell endogenous tumor-specific promoter. Preferably, the DNA construct comprises the upstream and downstream regions homologous to the AFP promoter and gene locus. Using standard gene targeting techniques, for example as described in A. L. Joyner's Gene Targeting: A Practical Approach, 1993, Oxford University Press, a single-copy of the exogenous C/EBPα transgene is placed under the control of the endogenous human or animal tumor-specific promoter (ex. AFP promoter) to create a mammalian host cell or a knock-in non-human animal capable of producing exogenous C/EBPα at a time different from the endogenous C/EBPα. For example, when the exogenous C/EBPα transgene is placed under the control of the endogenous AFP gene to create, for example, a C/EBPα gene knock-in mouse strain, the exogenous C/EBPα is transcribed specifically in the fetal liver at a time point earlier than normal as well as in developing liver tumor cells.

According to another aspect, the invention relates to a host cell, preferably a eukaryotic cell, comprising the DNA construct or any expression vector according to the invention. The host cell is preferably a mammal, human or animal cell and more preferably, a hepatocyte.

The host cell transfected with the DNA construct or expression vector of the invention is indicated as a tumor-resistant cell.

Accordingly, the invention provides tumor-resistant cells in which the exogenous C/EBPα transgene is specifically transcribed by the endogenous tumor-specific promoter, which is reactivated in tumors (where the normal expression of endogenous C/EBPα is turned off) initiating transcription of exogenous C/EBPα and inhibiting cell proliferation. Accordingly, the exogenous C/EBPα introduced into the host cells with the DNA construct is only produced in transfected hepatocytes rendering them less prone to tumorigenesis and carcinogenesis than normal hepatocytes. Such tumor-resistant cells are useful for transplant or therapy. For example, hepatocytes containing the C/EBPα knock-in allele could be used to provide transplant patients with a cell source that is more resistant to tumorigenesis than normal liver cells and thus give the patient a level of protection against future liver cancer. Additionally, the tumor-resistant hepatocytes also can be used to populate bioartificial liver systems. The tumor-resistant hepatocytes in such a system would be less likely to secrete harmful tumorigenic substances into the body of the patient than non-transfected hepatocytes in traditional bioartificial systems.

The tumor-resistant host cell of the invention may be a single cell, a cell culture, a cell aggregate or a cell graft for transplant.

The tumor-resistant host cell may also be isolated from knock-in animals modified by the DNA construct or expression vector of the invention as disclosed below.

The tumor-resistant host cell may also be isolated from human or animal pluripotent embryonic (ES) or adult stem cells transfected with the DNA construct of expression vector. Such transfected embryonic or adult stem cells may be used in therapy to regenerate a tissue or organ in need of transplant or regeneration. Any ES or adult stem cells known in the art can be used. They can be, for example, human or animal ES or adult stem cells, of a normal karyotype and capable of continued proliferation in an undifferentiated state or capable of differentiating into a specific tissue cell. For example, those described in U.S. Pat. No. 5,843,780, U.S. Pat. No. 6,200,806, US2001024825 or US2003008392 (herein incorporated by reference). Also Thomson J., et al., 1995, Proceedings of the National Academy of Sciences of USA, Vol.92, No.17:7844-7848; Reubinoff B. E. et al., April 2000, Nature Biotechnology, Vol.18, pages 399-404 (both of them herein incorporated by reference).

They can also be used for the preparation of transgenic animals, as explained below.

The invention provides a mammalian, human or animal, hepatocyte graft for administration to an area in need of transplant, wherein the cells of the hepatocyte graft are tumor-resistant cells and comprise the DNA construct of the invention. The graft may be an autograft or allograft.

Also provided is a process for preparing a hepatocyte graft for administration to an area in need of transplant, wherein the process comprises making the hepatocytes of the graft tumor-resistant by transfecting these hepatocytes with the DNA construct of the invention.

Further, it is provided a method for transplanting hepatocytes, hepatocyte aggregates or hepatocyte grafts, comprising preparing hepatocytes, hepatocyte aggregates or hepatocyte grafts modified with the DNA construct of the invention, and transplanting the modified prepared hepatocytes, hepatocyte aggregates or hepatocyte grafts to an area in need of transplant.

According to another aspect, it is provided a method of treating cancer in human or animals whose cancers produce elevated levels of AFP comprising treating the human or animal cancer with the DNA construct of the invention. In fact, the inverse relationship in gene activation between C/EBPα and AFP presents an ideal solution for artificially expressing C/EBPα under the regulatory elements of the AFP gene to suppress hepatic tumor growth.

Hepatocellular carcinoma (HCC) is one of the most common cancers in the world and its prognosis remains poor (Nakatsura T., et al., 2003, Biochem Biophys Res Commun., 306(1):16-25). Because of insufficient quantities of transplantable livers, alternatives for intact tissue transplantation have been developed including cellular hepatocyte transplantation (Ohashi K., et al., 2001, J Mol Med, 79:617-630) and, for short durations, the use of bioartificial livers. A major concern regarding these alternatives is the potential of the cells in these systems to undergo neoplastic transformation. Transplanted hepatocytes that become malignant pose obvious dangers to the recipient; malignant cells in bioartificial livers also present serious problems as tumorigenic factors may be secreted by these cells into the body of the patient. To reduce these risks in hepatocyte transplantation and bioartificial liver systems, genetic modifications in hepatocytes that minimize the occurrence of tumorigenesis are highly desirable.

According to a further aspect, it is therefore provided tumor-resistant hepatocyte cultures or hepatocyte aggregates prepared according to any embodiment of the invention for use in bioartificial livers (also known as hepatocyte bioreactors).

Hepatocyte bioreactors or bioartificial livers are known in the art (for example as disclosed in U.S. Pat. No. 5,270,192 herein incorporated by reference).

A hepatocyte bioreactor or bioartificial liver may comprise a containment vessel having a perfusion inlet and a perfusion outlet; a matrix within the containment vessel so as to entrap hepatocyte aggregates within the containment vessel while allowing perfusion of the matrix. Examples of bioartificial livers known in the art are: Extracorporeal Liver Assist Device (ELAD®), Vitagen; HepatAssist 2000, Circle Biomedical; Bioartificial Liver Support System (BLSS®), Excorp Medical, Inc.; LIVERX2000 System, Algenix Inc.; Modular Extracorporeal Liver System (MEL®), Charite Virchow Clinic Berlin, etc.

Tumor-resistant host cells, preferably hepatocytes, transfected with the DNA construct of the invention are preferably isolated from knock-in animals prepared as described below.

Experimental results using cultured primary liver cells derived from newborn knock-in mice indicate that the cells experience a significantly greater degree of growth inhibition than normal cells when exposed to chemically induced DNA damage. DNA damage is one of the initiators of HCC (Bralet M., et al., 2002, Hepatology, 36(3):624-630). This observation suggests that the cultured knock-in liver cells possess a greater resistance to tumorigenesis than normal liver cells.

Another embodiment of the invention is to provide a method for the treatment of symptoms in premature human infants or premature animals associated with liver glycogen-insufficiency.

The capacity to synthesize and store glycogen is one of the hallmarks of mature hepatocytes. Normal hepatocytes do not gain this ability until near the time of birth. As described later, the present authors have found that after birth, the C/EBPα gene knock-in mice develop completely normally and exhibit no adverse effects from carrying the C/EBPα transgene. Accordingly, if the accelerated maturation of hepatocytes is introduced into prematurely born human infants or prematurely born non-human animals, a large portion of the symptoms related to energy insufficiency in the premature infants or animals would be alleviated. This goal is achieved by administering to, injecting into, or transfecting the liver of a premature infant or animal, showing symptoms associated with liver glycogen-insufficiency, with the AFP-C/EBPα construct of the invention. Transient transfection of the DNA construct into premature infants or animals accelerates the maturation process of their underdeveloped livers. Furthermore, as activation of the AFP promoter is specific to the fetal liver, inadvertent transfection of non-liver cells would not result in unwanted C/EBPα expression.

Also, the AFP-C/EBPα construct of the invention can be used in a new gene therapy strategy to combat hepatocellular carcinoma in humans or animals.

Transient transfection of the DNA construct into patients with liver cancer would suppress growth only of cancerous cells without affecting normal cells because the majority of liver cancer cells reactivate the AFP promoter while normal cells do not.

In order to provide a “gene therapy” for the cancer, the DNA construct of the invention is preferably introduced into a suitable vector, most preferably, viral or retroviral vectors are employed for this purpose. Examples of suitable vectors, in addition to those identified above, are discussed by Fletcher, F. A. et al., 1991, J. Exper. Med. 174:837-845; M akel a, T. P. et al. ,1992, Gene 118:293-294; Porgador, A. et al., 1992, Canc. Res. 52:3679-3686; Yoshimura, K. et al., 1992, Nucl. Acids Res. 20:3233-3240.

The principles of gene therapy are disclosed by Oldham, R. K. (In: Principles of Biotherapy, Raven Press, NY, 1987), and similar texts. Disclosures of the methods and uses for gene therapy are provided by Boggs, S. S. (Int. J. Cell Clon. 8:80-96 (1990)); Karson, E. M. (Biol. Reprod. 42:39-49 (1990)); Ledley, F. D. (In: Biotechnology, A Comprehensive Treatise, volume 7B, Gene Technology, VCH Publishers, Inc. NY, pp 399-458 (1989)); all of which references are incorporated herein by reference. Such gene therapy can be provided to a recipient in order to treat (i.e. suppress or attenuate) an existing condition, or to provide a prophylactic gene therapy to individuals who, due to inherited genetic mutations, or somatic cell mutation, contain cells having impaired gene expression (for example, only a single functional allele of the C/EBPα gene).

Adenovirus vectors may be employed to deliver the exogenous C/EBPα-encoding sequences to the liver (as by injecting such vectors directly into the recipient's portal vein). The nature of the blood flow to the liver makes it likely that any vector delivered via the portal vein will come to reside within the hepatocytes and tumors of the liver, but is unlikely to extend outside of that organ to other tissues. In sum, the vectors are likely to become “trapped” in the liver tissue which serves as a filter for the body.

In a similar manner as above, constructs containing a lung-specific promoter in place of the AFP promoter can be used to accelerate maturation of underdeveloped lung tissues leading to improved survival and quality of life for these infants as well as to treat pulmonary cancer in adult human or animals. Some promoters which may be used in conjunction with C/EBPα for inhibiting lung cancer are: GRP (gastrin-releasing peptide), NSE (neuron specific enolase), and CEA (carcinoembryonic antigen).

Another embodiment of the invention relates to the preparation of knock-in non-human animals, preferably mammals, transfected with the DNA construct or expression vector of the invention.

The present inventors have successfully used genetic engineering processes to create a C/EBPα gene knock-in mouse model that expresses the C/EBPα transgene specifically in liver cells in a bid to inhibit the development of hepatocellular carcinoma. Standard gene targeting techniques (as described in A. L. Joyner's Gene Targeting: A Practical Approach, 1993, Oxford University Press) enabled the present authors to place a single copy of the C/EBPα transgene under the regulation of one of the two endogenous alleles of the mouse AFP gene promoter. However, as already mentioned, other tumor-specific promoters other than the AFP promoter may be used. Also other knock-in non-human animals other than mice can be created.

Knock-in non-human animals can be prepared according to any standard technique known in the art.

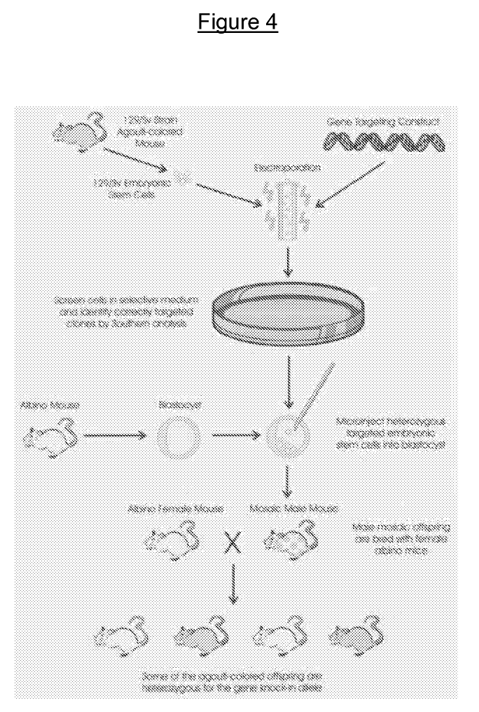

An outline of an example of the preparation of knock-in mice is shown in FIG. 4.

The DNA construct or expression vector according to any embodiment of the invention can be used for the preparation of non-human knock-in animals. Preferably, the DNA construct or vector is linearized and introduced into pluripotent recipient cells (preferably embryonic stem (“ES”) cells), or equivalent (Robertson, E. J., In: Current Communications in Molecular Biology, Capecchi, M. R. (ed.), Cold Spring Harbor Press, Cold Spring Harbor, N.Y. (1989), pp. 39-44, which reference is incorporated herein by reference). The transfected pluripotent cell may be cultured in vivo, in a manner known in the art (Evans, M. J. et al., Nature 292:154-156 (1981)) to form a transgenic animal.

Any ES cell may be used in accordance with the present invention. It is, however, preferred to use primary isolates of ES cells. For example, those prepared according to U.S. Pat. No. 5,843,780, U.S. Pat. No. 6,200,806, US2001024825 or US2003008392 (herein incorporated by reference). Also Thomson J., et al., 1995, Proceedings of the National Academy of Sciences of USA, Vol.92, No.17:7844-7848; Reubinoff B. E. et al., April 2000, Nature Biotechnology, Vol.18, pages 399-404 (both of them herein incorporated by reference).

Such isolates may also be obtained directly from embryos such as the CCE cell line disclosed by Robertson, E. J., In: Current Communications in Molecular Biology, Capecchi, M. R. (ed.), Cold Spring Harbor Press, Cold Spring Harbor, N.Y. (1989), pp. 39-44), or from the clonal isolation of ES cells from the CCE cell line (Schwartzberg, P. A. et al., Science 246:799-803 (1989), which reference is incorporated herein by reference). Such clonal isolation may be accomplished according to the method of E. J. Robertson (In: Teratocarcinomas and Embryonic Stem Cells: A Practical Approach, (E. J. Robertson, Ed.), IRL Press, Oxford, 1987) which reference and method are incorporated herein by reference. The purpose of such clonal propagation is to obtain ES cells which have a greater efficiency for differentiating into an animal.

The DNA construct comprising the exogenous C/EBPα may be introduced into the pluripotent cell by any method which allows the introduced molecule to undergo recombination at its regions of homology.

After electroporation, the ES cells are cultured in medium that selects for transfectants that have received and integrated the positive selectable marker gene sequence. The culturing conditions are then altered, such that they can select against recipient cells that have acquired the negative selection gene.

The survivors of such selection are then screened either for the function of the chromosomal allele, or more preferably, by Southern blot analysis for clones in which the selectable marker gene sequence has been introduced into the originally present chromosomal allele.

After selection for cells which have incorporated the desired DNA molecule (for example by selection for G418 resistant cells), the cells are cultured, and the presence of the introduced DNA molecule encoding exogenous C/EBPα under the control of the endogenous tumor-specific promoter is confirmed as described above. Any of a variety of methods may be used to identify cells which have undergone the desired recombination event. Direct screening of clones, use of PCR, use of hybridization probes, etc., may all be employed for this purpose.

ES cells that have undergone recombination may be used to produce the knock-in non-human transgenic animals. The ES cells are preferably cultured on stromal cells (such as STO cells (especially SNC4 STO cells) and/or primary embryonic fibroblast cells) as described by E. J. Robertson (In: Teratocarcinomas and Embryonic Stem Cells: A Practical Approach, (E. J. Robertson, Ed.), IRL Press, Oxford, 1987, pp 71-112), which reference is incorporated herein by reference. The stromal (and/or fibroblast) cells serve to eliminate the clonal overgrowth of abnormal ES cells.

In a preferred embodiment, the present authors have used gene targeting techniques to place a single-copy C/EBPα transgene under the control of the endogenous mouse α-fetoprotein (AFP) gene promoter to create a C/EBPα gene knock-in mouse strain. The transgene is expressed in the fetal liver several days prior to endogenous C/EBPα production and then shuts down at the time of birth in accordance to AFP regulation.

The experiments have demonstrated that early activation of C/EBPα in the knock-in mouse liver induces accelerated maturation of the tissue leading to glycogen deposition earlier than that found in wildtype mice. Accordingly, the transient transfection of C/EBPα-expressing constructs of the invention can be used in gene therapy to ameliorate the physiologic dysfunction of the human premature infant or premature animals.

Additionally, C/EBPα is known to be necessary for maintaining the non-transformed hepatic phenotype. Because the AFP promoter is reactivated in hepatocellular carcinoma, where normal C/EBPα expression is turned off, our knock-in mice gave us a unique opportunity to test C/EBPα as an inhibitor of liver tumor formation in vivo.

This novel non-human animal knock-in model may be used as an in vivo platform for identifying the types of carcinogens and the varieties of tumors that can be inhibited and suppressed by expression of transgenic C/EBPα.

More in particular, these novel non-human animal knock-in model or host cells, preferably hepatocytes or pneumocytes obtained from such knock-in animal, may be used as an in vivo platform for identifying the types of carcinogens and the varieties of tumors that can be inhibited and suppressed by expression of transgenic C/EBPα.

The host cells, in particular hepatocytes or pneumocytes, derived from this knock-in non-human animal may also be used as an in vitro assay for carcinogens that are susceptible to C/EBPα treatment.

Carcinogens are generally indicated as substances that increase the risk of initiating development of neoplasms in humans or animals. A carcinogen may be a chemical, a form of electromagnetic radiation or an inert solid body. Both genotoxic chemicals, which affect DNA directly, and nongenotoxic chemicals, which induce neoplasms by other mechanism, are included. DEN is an example of a genotoxic agent known to produce hepatocellular carcinomas (Bralet, M., et al., 2002, Hepatology 36(3):624-630).

Accordingly, it is also provided a method for in vivo screening and/or modulation of tumors comprising administering one or more carcinogens to a non-human animal modified with the DNA construct of the invention and identifying the tumors which are inhibited or suppressed by the expression of transgenic C/EBPα.

The knock-in non-human animal according to the invention may be used to supply liver cells that are highly resistant to tumorigenesis for use in models testing hepatocyte transplantation and for populating bioartificial liver systems. One common concern regarding both hepatocyte transplantation and bioartificial livers is the potential of their cells to undergo neoplastic transformation. The presence of the C/EBPox transgene in the hepatocytes would greatly reduce the risk of this complication.

Accordingly, it is provided: a non-human knock-in animal modified with the DNA construct or expression vector of the invention; a non-human knock-in animal, wherein the animal's endogenous tumor-specific promoter transcribes the exogenous C/EBPα transgene specifically in the fetal liver at a point earlier than normal; a knock-in animal, wherein the tumor-specific promoter is AFP; a knock-in animal, wherein the exogenous C/EBPα transgene expresses C/EBPα mRNA prior to endogenous C/EBPα production; a tumor-resistant cell isolated from the knock-in animal according to the invention; a tumor-resistant cell isolated from the knock-in animals, wherein the tumor-resistant cell is a hepatocyte or pneumocyte; a human or animal pluripotent embryonic or adult stem cell transfected with the DNA construct of claim; a tumor-resistant cell, wherein the tumor-resistant cell is isolated from a human or animal pluripotent embryonic or adult stem cell transfected with the DNA construct; a method for preparing a knock-in non-human animal comprising introducing the DNA construct or expression vector of the invention into a non-human animal pluripotent embryonic or adult stem cell, and breeding the cell culture.

The present invention also relates to a medical or pharmaceutical preparation comprising any DNA construct or expression vector of the invention and optionally a pharmaceutically acceptable carrier and/or diluent.

Said medical or pharmaceutical preparation can be used for any medical treatment, and in particular for any of the treatments indicated, such as but not limited to: the treatment of cancer, preferably for the treatment of cancer in a human or animal whose cancer cells produce elevate levels of AFP; the treatment of symptoms associated with liver glycogen-insufficiency in a human premature infant or a premature animal; the treatment of conditions associated with underdeveloped lung tissue in a human or animal.

The invention further relates to the use of any DNA construct or expression vector according to the invention for the preparation of a medical or pharmaceutical composition for the treatment of (but not limited to): the treatment of cancer, preferably for the treatment of cancer in human or animal whose cancer cells produce elevate levels of AFP; the treatment of symptoms associated with liver glycogen-insufficiency in a human premature infant or a premature animal; the treatment of conditions associated with underdeveloped lung tissue in human or animal.

Having now generally described the invention, the same will be more readily understood through reference to the following examples which are provided by way of illustration, and are not intended to be limiting of the present invention.

### EXAMPLES

### Example 1

Method Used to Produce the C/EBPα Gene Knock-in Mouse Stratagene's Mouse Lambda Genomic Library—129SvJ (Catalog #946313) was used to isolate the necessary AFP and C/EBPα sequences. Routine molecular biology techniques (for example Sambrook J. and Russel D. W., Molecular Cloning, A Laboratory Manual, 3^{rd} edition, 2001, Cold Spring Harbor Laboratory Press) were used to build the AFP-C/EBPα gene targeting construct detailed in FIGS. 1-3.

A 5.5 kb EcoRI-TaqI fragment of the AFP promoter and upstream sequence was spliced onto the NruI-SpeI C/EBPα gene sequence in a pBlueScript vector. An extra T nucleotide at position 5549 was retained between the AFP fragment and the C/EBPα fragment which facilitated the construction process. An oligonucleotide sequence containing the HinDIII identification tag and two polyadenylation signals was ligated into the SpeI site at the end of the C/EBPα gene sequence from position 7021-7056. A PGKneo expression cassette containing the neomycin gene and a PGK promoter (purchased from Lexicon Genetics Incorporated) was placed downstream of the C/EBPα polyadenylation signals by blunt-end ligation into an EcoRI site that was engineered into the identification tag oligonucleotide sequence. An additional 900 bp of AFP sequence comprising a portion of intron 1 through intron 3 isolated by SacI digestion was blunt-end ligated downstream of the neomycin cassette to provide sufficient homologous sequence to promote efficient gene targeting into the AFP locus. MC1 tk containing the thymidine kinase gene and MC1 promoter (purchased from Lexicon Genetics Incorporated) was placed at the 3′ end of the cloned sequence again by blunt-end ligation into the original 3′ end Sacd site. The obtained construct (25 μg/ml) was linearized using EcoRI and electroporated into mouse embryonic stem cells (1.8×10⁶ cells/ml) (as adequately explained in A. L. Joyner's Gene Targeting: A Practical Approach, 1993, Oxford University Press and Hogan et al.'s Manipulating the Mouse Embryo: A Laboratory Manual, 1994, CSHL Press). Positive drug selection using G418 (active ingredient, 0.18 mg/ml) and negative selection using gancyclovir (2 μmol/L) were employed to enrich the yield of gene targeted clones. Correctly modified stem cells were identified by PCR and Southern blotting. These cells were then used to generate chimeric founder mice carrying the altered allele in their germ cells. The founders were bred to produce gene targeted knock-in mice.

The complete process for generating the knock-in mice is shown in FIG. 4.

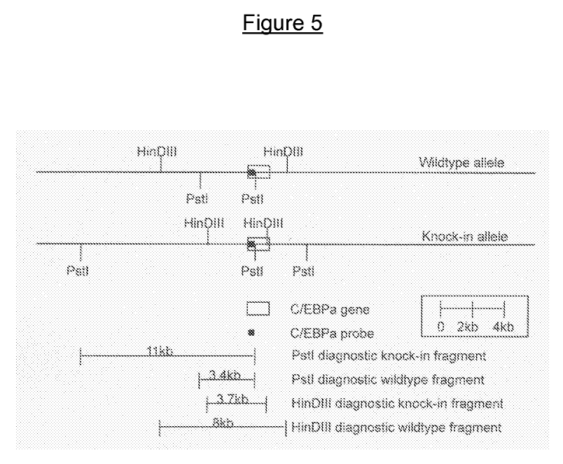

The wildtype C/EBPα gene locus and the expected knock-in gene locus are illustrated in FIG. 5. A C/EBPα probe would be expected to detect DNA fragments of different sizes in wildtype versus knock-in mice (as illustrated).

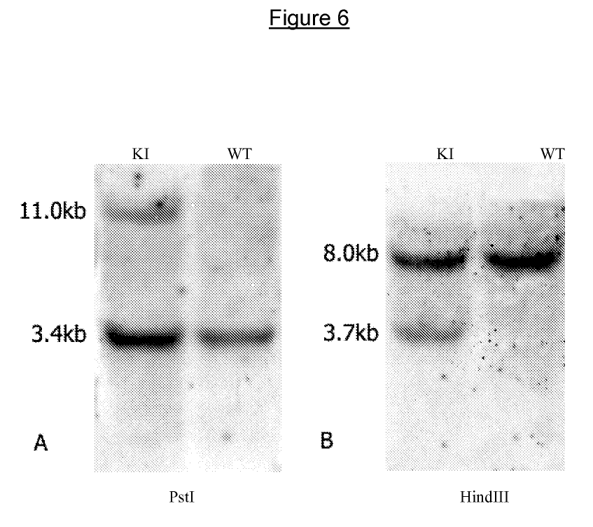

Southern blots probed with a C/EBPα sequence demonstrated the presence of the altered allele in the knock-in mice (FIG. 6). Under PstI digestion, knock-in mouse DNA shows an extra 11 kb C/EBPα fragment that is absent in the wildtype animal. Under HindIII digestion, the extra C/EBPα fragment is 3.7 kb in size.

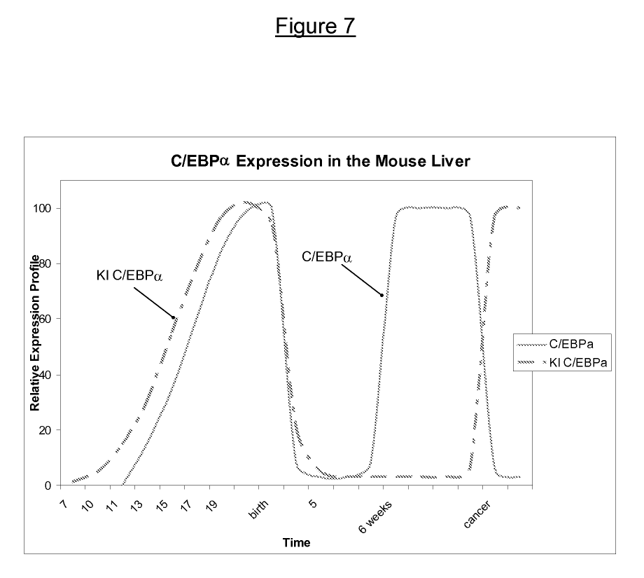

As the genetic modification was designed to place a single copy of the C/EBPα transgene under the regulation of one of the two endogenous alleles of the mouse α-fetoprotein (AFP) gene promoter, we considered that the transgene would yield an expression pattern like the one illustrated in FIG. 7.

### Example 2

Determination of Levels of C/EBPα mRNA and Glycogen Synthase mRNA

The levels of fetal C/EBPα mRNA have been assessed by using real-time PCR in a LightCycler. Oligonucleotide primers used were 5′-TGGACAAGAACAGCAACGAGTA-3′ (SED ID NO:3) and 5′-GCAGTTGCCATGGCCTTGA-3′ (SEQ ID NO:4).

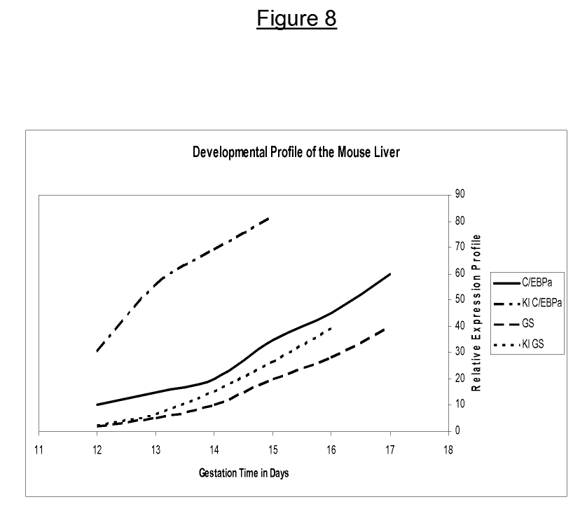

We found C/EBPα mRNA in the knock-in mice to be detectable earlier and at higher amounts than in the wildtype animals (FIG. 8).

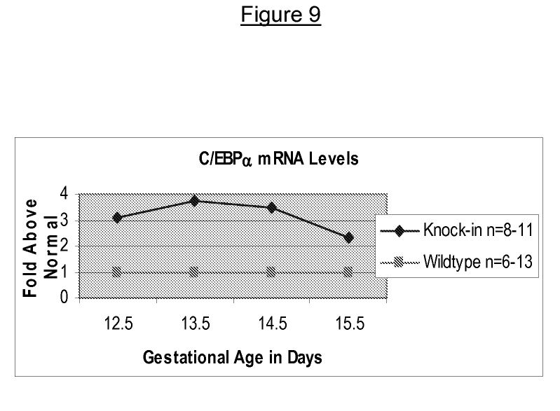

Additionally, to evaluate the functionality of the newly expressed transgene, we measured the levels of glycogen synthase mRNA also using real-time PCR. Oligonucleotide primers used were 5′-CCATGAACAGCAAGGGTTGTAA-3′ (SEQ ID NO:5) and 5′-TGGAAGTGGGCAACCACATA-3′ (SEQ ID NO:6). C/EBPα mRNA levels were determined to be between 100% to 300% higher in the knock-in mice from 12.5-15.5 days of gestation than in the wildtype and glycogen synthase mRNA was 25%-50% higher over the same time period (FIG. 9).

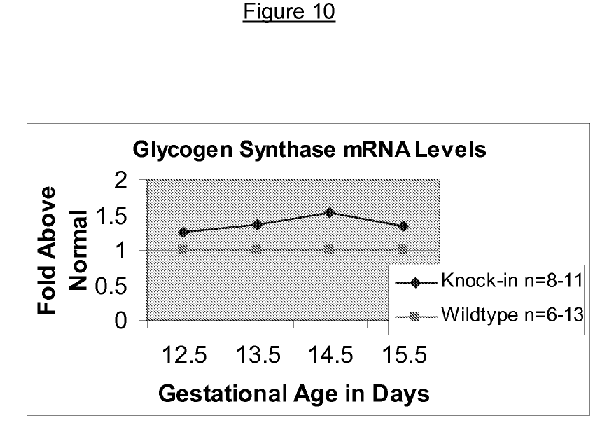

Earlier studies had shown C/EBPα to be a transcriptional activator of the glycogen synthase gene (Wang, N. D., et al., 1995, Science 269:1108-1112). Glycogen synthase mRNA was found to be expressed at higher levels than in the wildtype mice (FIG. 10).

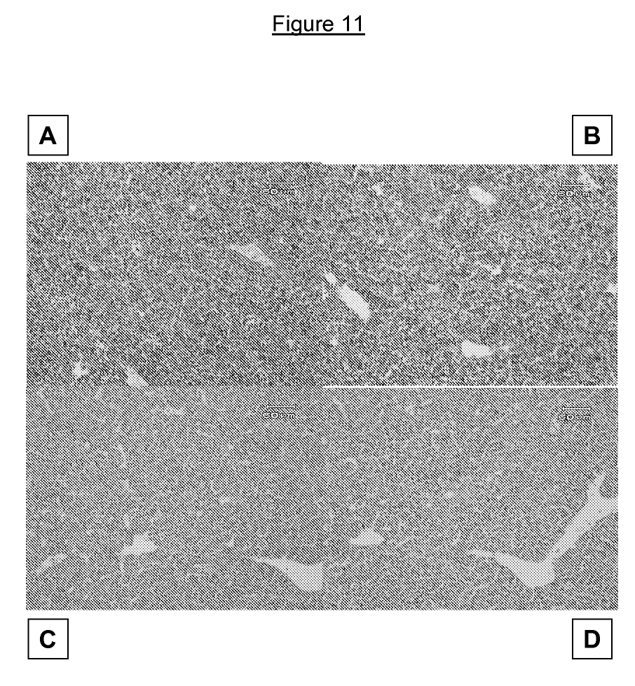

Definitive proof of the functionality of the transgene product came in the form of histology sections of the livers from the mice. Periodic acid-Schiff (PAS) and PAS-diastase treated slides showed the presence of glycogen in the knock-in mice on day 15.5 of gestation when wildtype mice had not yet begun to deposit liver glycogen (FIG. 11). Under PAS staining, glycogen produces a pinkish hue, which is seen as darker cells in FIG. 11A. FIG. 11A shows that glycogen is present in the liver of the knock-in mouse fetus at 15.5 days gestational age. Diastase treatment digests away any glycogen that may be present on the slide. FIG. 11B shows that diastase treatment followed by PAS staining does not produce the pink colour in the knock-in mouse liver indicating that the pink colour had indeed originated from liver glycogen. FIGS. 11C (PAS staining) and 11D (PAS-diastase) show that wildtype fetal livers at 15.5 days gestational age do not normally contain detectable glycogen.

### Example 3

Resistance to Tumorigenesis

We further verified that our genetic modification could confer upon hepatocytes a degree of resistance to tumorigenesis.

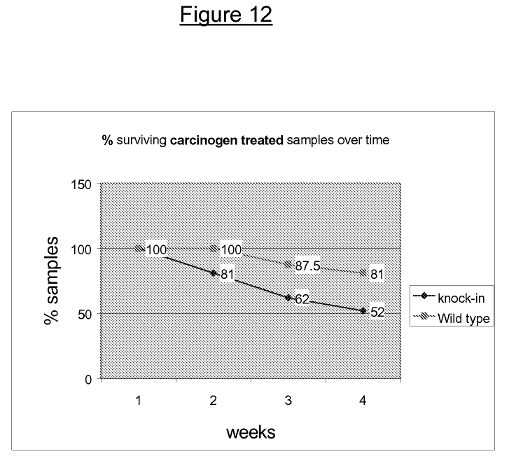

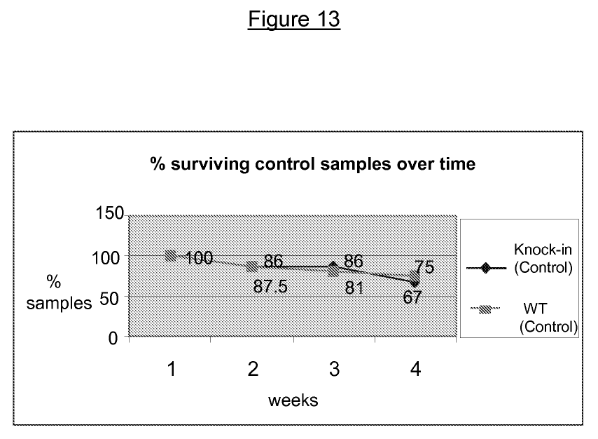

We exposed cultures of primary hepatocytes derived from 18.5 day old mouse fetuses to 3.2 mM n-nitrosopiperidine, a genotoxic agent. Fetal livers were minced with a scalpel under sterile conditions in calcium-free Earle's Balanced Salt Solution (Gibco-BRL) containing 50 mmol/L EDTA to obtain tissue fragments. These tissue fragments were then incubated in Earle's Balanced Salt Solution with calcium (Gibco-BRL) and 0.02 mg/ml soybean trypsin inhibitor (Sigma) and 1.6 mg/ml collagenase (Sigma) and incubated at 37° C., 5% CO₂. The EDTA/collagenase treatments were performed twice. For long-term observation, the control cells were grown in Hepatocyte Culture Medium (HCM) (Clonetics) and the experimental groups were grown in HCM with 3.2 mM n-nitrosopiperidine (Sigma) dissolved directly into it. Our experiments showed that the cultures of the knock-in hepatocytes experienced a greater degree of growth inhibition and cell death than the wildtype cells (FIGS. 12). FIG. 13 shows that, in the absence of carcinogen, the knock-in cells demonstrated survival rates comparable to that of wildtype cells.

Lastly, after a year-long experiment in which twelve day old juvenile knock-in and wildtype mice were given intraperitoneal injections of 10 μg diethylnitrosamine (DEN) per gram of body weight, we were able to observe the effects of our transgene on the development of liver tumors in live animal models. DEN is another genotoxic agent known to produce hepatocellular carcinomas (Bralet, M., et al., 2002, Hepatology 36(3):624-630).

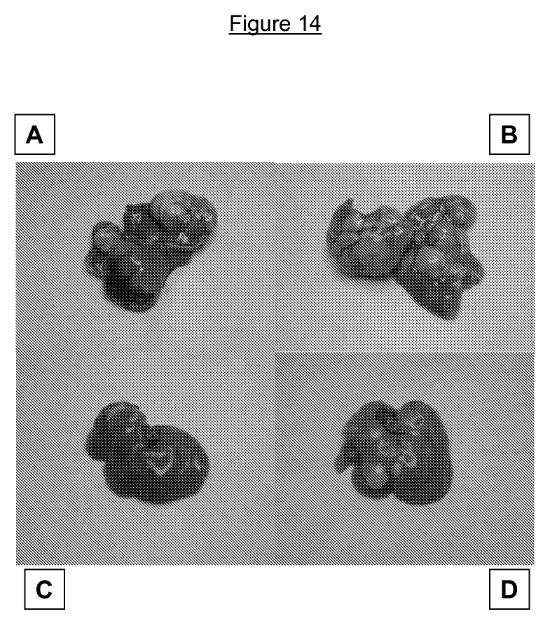

The mice were maintained on a 12 hour light and 12 hour dark daily cycle and had unrestricted access to food and water. FIG. 14 shows that while the wildtype livers (A, B) contained many tumorous growths, knock-in livers (C, D) had dramatically fewer tumors. A visual survey of the liver surfaces for nodules and tumors revealed markedly fewer numbers in the knock-ins (19.8±12.6; n=10) than the wildtypes (41.0±23.3; n=5) (Student t test, P>0.05). These data provide compelling evidence that C/EBPα expressed under the regulation of the AFP promoter results in liver cells that are much less likely to undergo tumorigenesis than normal hepatocytes.

1. A DNA construct comprising:
a first region having homology with an endogenous tumor-specific promoter gene sequence of a gene locus;
a DNA molecule encoding C/EBPα, the DNA molecule located 3′ of the first region; and
a second region having homology with a region within the gene locus, the second region located 3′ of the DNA molecule;
wherein the construct is operable to express a functional C/EBPα protein and wherein the endogenous tumor-specific promoter gene sequence and the C/EBPα have an inverse relationship in gene activation.

2. The construct of claim 1, wherein the DNA molecule encodes human C/EBPα.

3. The construct of claim 1, wherein the DNA molecule encodes mouse C/EBPα.

4. The construct of claim 1, wherein the tumor-specific promoter gene comprises the AFP promoter gene and the gene locus comprises the AFP gene locus.

5. The construct of claim 1, further comprising a selectable marker, an identification tag, or a combination thereof.

6. An expression vector comprising the construct of claim 1.

7. An expression vector comprising a construct which comprises:
a first region having homology with an endogenous tumor-specific promoter gene sequence of a gene locus;
a DNA molecule encoding the C/EBPα, the DNA molecule located 3′ of the first region; and
a second region having homology with a region within the gene locus, the second region located 3′ of the DNA molecule;
wherein the construct is operable to express a functional C/EBPα protein;
the vector further comprising:
a DNA molecule having the sequence of SEQ ID NO:1; and
a DNA molecule having the sequence of SEQ ID NO:2.

8. A host cell comprising the construct of claim 1.

9. The host cell of claim 8, wherein the host cell is present in a cell culture.

10. The host cell claim 8, wherein the host cell is an animal hepatocyte or pneumocyte.

11. The host cell of claim 9, wherein the host cell is an isolated human cell.

12. The host cell of claim 8, wherein the host cell is a cell which has been made tumor resistant upon addition of said construct.

13. The host cell of claim 10, wherein the host cell is a hepatocyte suitable for grafting in an area in need of transplant.

14. A hepatocyte bioreactor comprising the construct of claim 1.
